(19) United States
(12) Patent Application Publication
(10) Pub. No.: US 2016/0030123 A1
(43) Pub. Date: Feb. 4, 2016

(54) TISSUE EXPANDER AND METHODS

(71) Applicant: Bin SONG, Beijing (CN)

(72) Inventor: BIN Song, Cleveland Heights, OH (US)

(21) Appl. No.: 14/774,968

(22) PCT Filed: Mar. 11, 2014
(86) PCT No.: PCT/CN2014/073194

§ 371 Date: Sep. 11, 2015

Related U.S. Application Data
(60) Provisional application No. 61/777,233, filed on Mar. 12, 2013.

(51) Int. Cl. A61B 19/00

(52) U.S. Cl. CPC A61B 19/24; A61F 2/12

(57) ABSTRACT

A tissue expander adapted for increasing overlying tissue surface area and size of an associated perforator vessel includes an inflatable bladder adapted to be temporarily implanted subcutaneously in a patient at a donor site. The bladder forms a recess for receiving therein a perforator vessel. Bladder volume is increased incrementally over time to increase overlying tissue surface area and size of the associated perforator vessel. The expanded tissue and/or the perforator vessel can be harvested for use as an island flap, a free flap, or a vascular graft.

### FIELD OF THE INVENTION

The invention relates generally to tissue expanders and associated surgical techniques and, more specifically, to tissue expanders designed to increase the tissue surface area supplied by a vascular pedicle, as well as the diameter, length and flow territory of the vascular pedicle.

### BACKGROUND OF THE INVENTION

Tissue expanders are inflatable balloon-like devices that are implanted beneath a patient's skin and gradually filled with saline to stretch the skin overlying it over a period of weeks and, therefore, increase its surface area. The newly developed excess skin envelope is used in plastic/reconstructive surgical procedures to reconstruct adjacent soft tissue deficiencies due to congenital conditions, traumatic injuries, skin cancers, etc. The size and shape of the expander is determined by the anatomic location where it is used and by the amount of tissue required. Conventional expanders are round, rectangular, elliptical or crescent-shaped. See, e.g., http://www.mentormedical.co.uk/tissue-expanders.php

Currently available tissue expanders are placed only with regard to the location and amount of expanded skin required and not with regard to the circulation which provides its nourishment. The expanded skin can be advanced or rotated in a very limited degree, since its blood supply is diffuse and comes randomly from a wide base. This severely limits the applicability of the harvested tissue, because any movement of the expanded tissue potentially compromises the circulation to the expanded tissue. For that reason these random advancement and rotation flaps can only repair defects that are immediately adjacent to the expanded tissue.

Unlike random flaps, island flaps have a dominant feeding vessel (termed axial vessels), with flap circulation based on the underlying vessel. An island flap typically can rotate up to 360 degrees allowing the donor site to be remote from the defect. There are a very limited number of areas in the body where tissue supplied by axial vessels is expendable and can be used as island flaps for defect reconstruction.

Accordingly, there exists a need to provide enhanced surgical techniques afforded by newly developed tissue expanders to increase the flexibility in treating a variety of skin defects, without the numerous limitations of conventional flap methodologies.

### SUMMARY OF THE INVENTION

The types of tissue expanders described herein are configured and adapted to be placed in relation to discrete arteries and veins located throughout the skin surface (termed perforator vessels) allowing expansion of both the perforating vessels and associated tissue territories. These expanded tissues can now function as island flaps, with all of the before described advantages over random flaps.

Another application of this invention is to generate an increase in vessel diameter and pedicle length of an expanded island flap. This also allows the potential for the expanded dominant feeding vessel and its concomitant vascular territory to be detached from its location and transferred to a remote location with its viability maintained by the microsurgical anastomosis of the vessels in the flap with vessels in the recipient area. This technique is sometimes referred to as a free tissue transfer; however, the number and locations of suitable donor sites using conventional techniques are quite limited.

In various aspects and embodiments, devices in accordance with the invention are adapted to effectively and efficiently expand a perforator vessel associated with a flap, in order to enlarge the associated vascular pedicle and its vascular territory sufficiently to allow subsequent use as an island flap.

According to one aspect of the invention, a tissue expander is adapted for increasing overlying tissue surface area and size of an associated perforator vessel. In one embodiment, the tissue expander includes an inflatable bladder adapted to be disposed subcutaneously and forming a recess for receiving therein in incidental contact a perforator vessel. The bladder may be of the self-expanding type or may include a port in fluidic communication with an interior volume of the bladder. The bladder may be manufactured from a flexible biocompatible material, such as an elastomer.

In certain embodiments, the bladder is adapted to inflate preferentially in a first zone relative to a second zone, for example by making the first zone with a first wall thickness and the second zone with a greater wall thickness. Alternatively or additionally, the first zone may be made with a material having a first durometer and the second zone with a material having a greater durometer. Depending on the configuration of the expander, the second zone may be disposed along at least a portion of the recess and/or along at least a portion of a perimeter of the bladder remote from the recess.

In some embodiments, the bladder or at least a portion thereof can be generally U-shaped, generally V-shaped, or generally C-shaped, with the recess being formed by a pair of arms extending from a common junction portion. When the bladder is generally U-shaped or C-shaped, the arms can be substantially parallel, although they can diverge in the case of a V-shaped bladder.

Depending on the particular application, the tissue expander can be any size. In one embodiment, the recess is sized to receive a perforator vessel in a region of the expander having a width of at least about 1 mm.

For those embodiments that include a port, the port can be disposed on a wall of the bladder or adapted to be disposed remotely from the bladder, for example being connected thereto with a tube of a flexible, semi-rigid or rigid nature.

In some embodiments, at least a portion of an exterior of the bladder may have a smooth surface, a textured surface, or a combination thereof, and optionally contoured edges.

In one embodiment, the bladder is configured with a length greater than a width, with the recess being formed as a slot disposed along a portion of a longitudinal centerline of the bladder. The bladder may be substantially symmetrical about a plane passing through the longitudinal centerline, substantially symmetrical about another plane, or substantially asymmetrical.

In certain embodiments, the tissue expander may include a support disposed along at least a portion of one side of the bladder. Alternatively or additionally, the tissue expander may include a retainer, such a one or more tabs optionally with reinforced apertures, adapted to be secured to adjacent body structure when the bladder is implanted, to reduce a likelihood of migration of the bladder.

According to another aspect, a method of expanding tissue for use by a patient includes implanting temporarily a tissue expander beneath a locus of skin and subcutaneous tissue supplied by an associated perforator vessel, wherein the perforator vessel is received at least partially in a recess formed in the tissue expander. The method further includes incrementally increasing an internal volume of the tissue expander over time to increase overlying tissue surface area and size of the associated perforator vessel. Increase of the size of the associated perforator vessel is incidental to increase of the surface area of the overlying tissue. In some embodiments, the tissue expander includes an inflatable bladder. The bladder may be of the self-expanding type or may include a port in fluidic communication with an interior volume of the bladder.

Once implanted, the volume increasing step can include adding sterile fluid to the internal volume of the bladder via the port. In certain embodiments, the bladder is made from a flexible biocompatible material, for example an elastomer. The volume increasing step may inflate the bladder preferentially in a first zone relative to a second zone, for example because the first zone has a first wall thickness and the second zone has a greater wall thickness and/or because the first zone has a first durometer and the second zone has a greater durometer. Depending on the particular configuration and application, the second zone may be disposed along at least a portion of the recess and/or may be disposed along at least a portion of a perimeter of the bladder remote from the recess.

In some embodiments, the bladder or at least a portion thereof can be generally U-shaped, generally V-shaped, or generally C-shaped, with the recess being formed by a pair of arms extending from a common junction portion. When the bladder is generally U-shaped or C-shaped, the arms can be substantially parallel, although they can diverge in the case of a V-shaped bladder.

Depending on the particular application, the tissue expander can be any size. In one embodiment, the recess is sized to receive a perforator vessel in a region of the expander having a width of at least about 1 mm.

For those embodiments that include a port, the port can be disposed on a wall of the bladder or adapted to be disposed remotely from the bladder, for example being connected thereto with a tube of a flexible, semi-rigid or rigid nature.

In some embodiments, at least a portion of an exterior of the bladder may have a smooth surface, a textured surface, or a combination thereof, and optionally contoured edges.

In one embodiment, the bladder is configured with a length greater than a width, with the recess being formed as a slot disposed along a portion of a longitudinal centerline of the bladder. The bladder may be substantially symmetrical about a plane passing through the longitudinal centerline, substantially symmetrical about another plane, or substantially asymmetrical.

In certain embodiments, the tissue expander may include a support disposed along at least a portion of one side of the bladder. Alternatively or additionally, the tissue expander may include a retainer, such a one or more tabs optionally with reinforced apertures, and include the step of securing the expander by suturing the retainer to adjacent body structure when the bladder is implanted, to reduce a likelihood of migration of the bladder.

In various embodiments, the method may include the steps of removing the tissue expander, harvesting at least a portion of the expanded tissue and the associated perforator vessel as a free flap, relocating the free flap to another location of the patient's body, and attaching the associated perforator vessel to vasculature of the patient.

Alternatively, the method may include the steps of removing the tissue expander, harvesting at least a portion of the associated perforator vessel, relocating the harvested vessel to another location of the patient's body, and attaching the harvested vessel as a vascular graft to vasculature of the patient.

Alternatively, the method may include the steps of, removing the tissue expander, harvesting at least a portion of the expanded tissue and the associated perforator vessel as an island flap, and relocating the island flap to a proximate location of the patient's body.

It is also contemplated that expanded tissue and/or perforator vessels may be harvested from a compatible donor for use in another patient.

### DETAILED DESCRIPTION

As discussed above, conventional tissue expanders are inflatable balloon-like devices that are implanted beneath a patient's skin and gradually filled with saline in order to gradually stretch the skin overlying it and therefore increase its surface area. The now excess skin envelope is used to reconstruct adjacent soft tissue deficiencies. The size and shape of the expander is determined by the anatomic location where it is used and by the amount of tissue required. The potential tissue expansion is determined by the volume and dimensions of the expander.

Conventional tissue expanders are placed only with regard to the location and amount of expanded skin required. The expanded skin, because it has no defined relation to a vascular pedicle (termed a random flap), can be advanced or rotated in a very limited degree, since its blood supply is diffuse and comes randomly from a wide base (termed a skin pedicle). Tissue expanders according to the instant invention can be advantageously located with respect to a vascular pedicle in the area to be expanded. This allows the expanded territory to function as an island flap.

The discrete vascular pedicle in an island flap allows much greater flexibility in movement and positioning of the flap, since the base of the flap is the vascular pedicle, which can be rotated virtually 360 degrees. There are only a few naturally occurring areas in the human body with vascular pedicles that supply expendable areas of soft tissue. Therefore, absent the teachings of the instant invention, there are only a few areas where the island flap concept can be employed. Methods and devices according to the instant invention that create island flaps in more locations are of tremendous utility and of great clinical usage. For example, there are many small vascular pedicles (termed a perforator vessel) supplying small areas of skin throughout the body. See FIG. A, below, from G. I. Taylor and J. H. Palmer: The vascular territories [angiosomes] of the body: experimental study and clinical applications. Br J Plast Surg 1987;40:113-141 at page 130, the entire disclosure of which is incorporated herein by reference in its entirety. FIG. A depicts as black dots or small circles the large number of dominant cutaneous perforators linked to various source arteries, as the perforators emerge from the deep fascia.

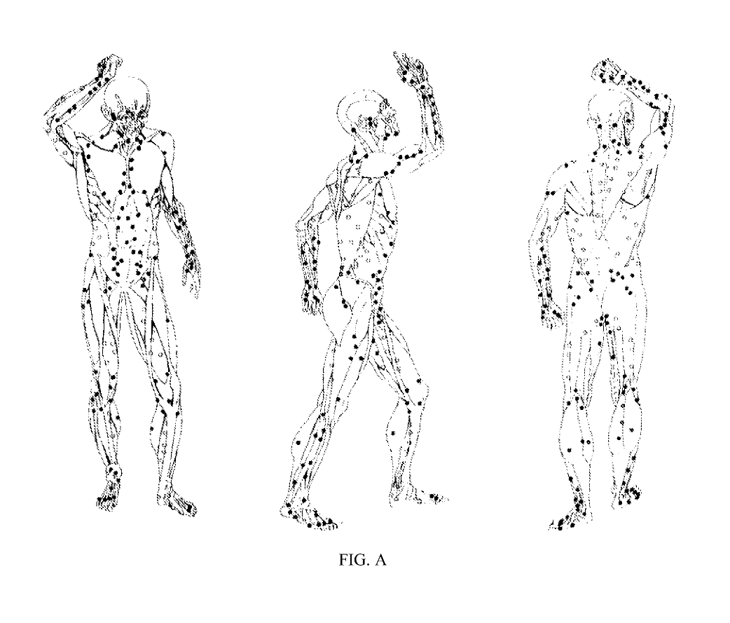

With this knowledge of the location of smaller perforator vessels, a new configuration tissue expander has been developed and configured with a recess formed in the bladder specifically to be placed beneath a perforating vascular pedicle and around the perforator vessel, typically on at least three sides. See FIGS. 1A-1C below, for one embodiment of the tissue expander. More specifically, FIG. 1A is a top or plan view of a perforator tissue expander in accordance with one embodiment of the invention. FIG. 1B is a lateral side view of the expander of FIG. 1A and FIG. 1C is a cross-sectional view of the expander of FIG. 1A.

This embodiment of the expander is a generally U-shaped bladder, having an overall generally rectangular dimensional volume when inflated defined by a length of about 130 mm, a width of about 60 mm, and a thickness or height of about 45 mm. The bladder is configured to inflate or expand primarily in the thickness or height direction after implantation, for example by injection of a sterile fluid such as sterile saline into the bladder through an injection port in fluidic communication with the interior volume of the bladder via a flexible tube. Inflation or expansion in other direction(s) is also contemplated. In the depicted embodiment, the tube is in a range of about 80 mm to about 100 mm in length. The port may be self-sealing and optionally located on the bladder, by including an integral magnetic location system, as known by those skilled in the art.

The bladder may be manufactured from any suitable flexible biocompatible material, such as silicone elastomer, having a nominal wall thickness in the range of up to about 0.2 mm to about 0.3 mm or greater. As noted in FIG. 1A, various zones of the bladder (e.g., Positions A and B) can be thicker than the nominal wall thickness, having a wall thickness of about double or triple or more, in the range of up to about 0.5 mm to about 0.7 mm or greater. Alternatively or additionally, these zones can be made of a material with a greater durometer or otherwise reinforced, to provide for preferential inflation of the bladder in thinner and/or softer zones.

In the depicted U-shaped embodiment, Position B is located at the end of the recess formed by a common junction portion of a pair of substantially parallel arms. The perforator vessel is disposed in the recess, generally proximate Position B, with the arms placed alongside. By reinforcing Position B, upon expansion of the bladder, the bladder wall is restrained from impinging on or pressing against the perforator vessel, in a manner that might constrict blood flow through the vessel. Alternatively or additionally, at least a portion of the perimeter of the bladder (depicted in this embodiment at Position A, remote from the recess) is similarly thickened or stiffened. This helps to prevent shifting or movement of the bladder and its arms during inflation in a manner that could cause impingement of the bladders and its arms against the perforator vessel.

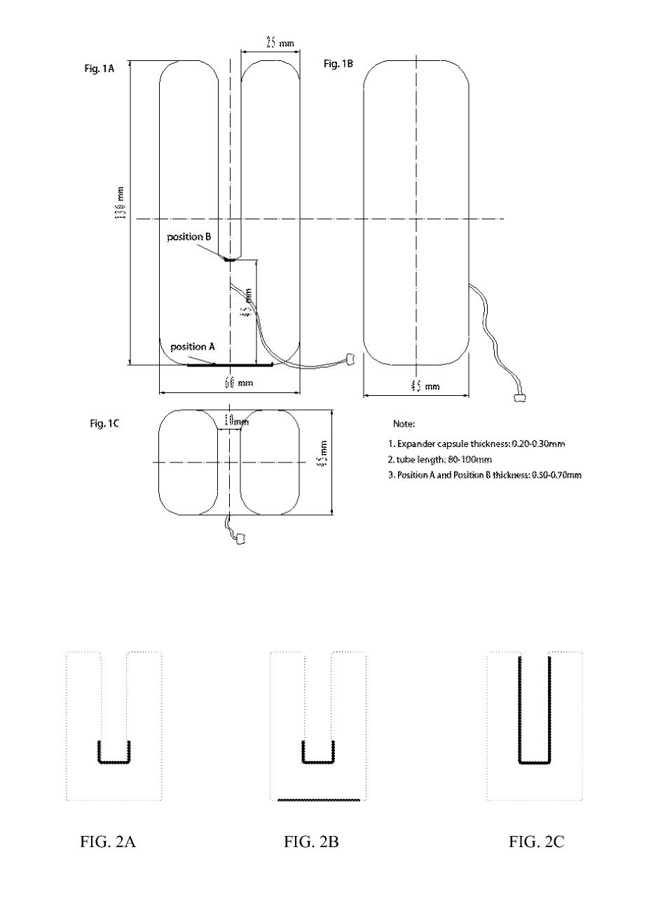

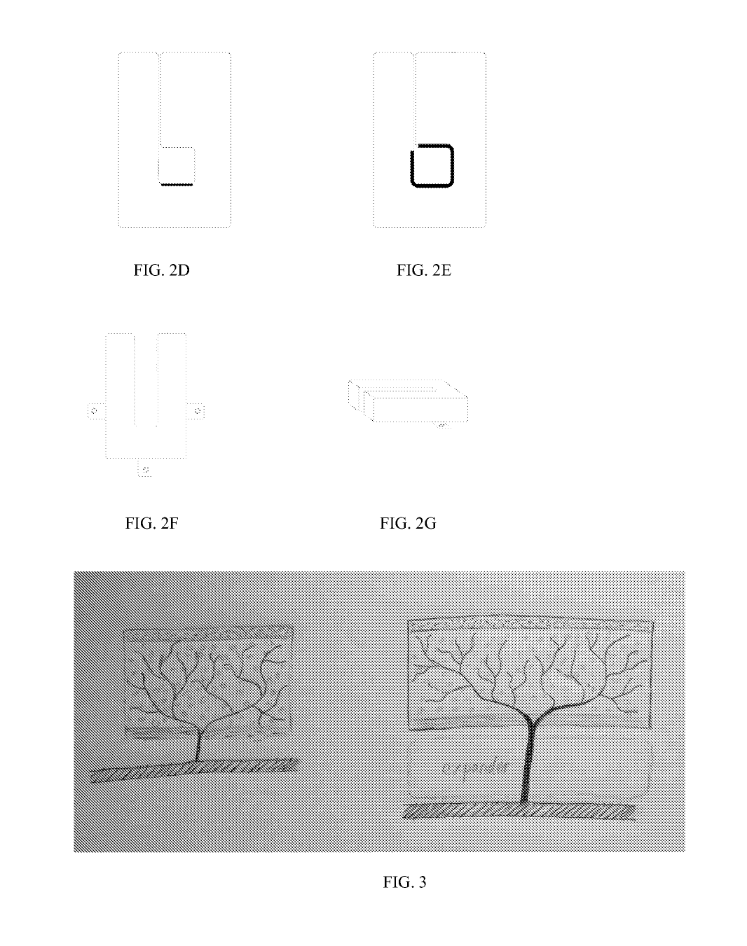

Naturally, other zones that those depicted can be addressed, to create the preferential expansion of the bladder to maintain the bladder in the desired orientation and configuration proximate the perforator vessel. See, for example, FIG. 2A, where a U-shaped zone at the end of the recess is reinforced.

In FIG. 2B, is a variant, combining reinforcement features in the recess and periphery from FIGS. 1A and 2A. In FIG. 2C, the entire longitudinal extent of the recess is reinforced.

FIGS. 2D and 2E depict a slightly asymmetrical bladder configuration, with the arms of the generally U-shaped bladder forming a relatively narrow entrance recess connecting to an enlarged end of the recess at the common junction portion. The reinforcement features at the end of the recess are limited to essentially one side in FIG. 2D; whereas, all sides are reinforced in FIG. 2E.

Referring back to the embodiment of FIG. 1A, the recess is about 10 mm wide and about 85 mm long, providing a significant clearance margin for growing the perforator vessel to a relatively large diameter, while substantially surrounding the vessel to encourage uniform growth. The relatively long recess permits axial movement of the bladder during the extended implantation period, without constriction of the vessel. Recess widths can be a minimum of about 1 mm, so as not to compromise blood flow, but can be about 5 mm, about 10 mm, or about 15 mm, up to about 20 mm or greater (e.g., 30 mm or 40 mm or more).

The surface of the bladder can be partially or wholly smooth, textured, or a combination of the two. Texturing can help to retain the bladder in place. To further limit movement or migration of the bladder, a retainer structure can be provided. For example, one or more tabs (optionally with reinforced apertures) can be disposed about the perimeter of the bladder or at any suitable location(s). See FIGS. 2F and 2G, below, for plan and perspective views of one such retainer structure. The retainer can be sutured to adjacent body structure when the bladder is implanted, to reduce a likelihood of migration of the bladder and impingement on the substantially circumscribed vessel. Additionally, the edges of the bladder can be radius or otherwise smoothly contoured, to minimize discomfort and the possibility of erosion.

To further control expansion of the bladder in desired direction(s), a support (e.g., a semi-rigid base structure or a thickened or a higher durometer material), may underlie or overlie some or all of the bladder. The support may be adapted to perform as the retainer, as well.

Accordingly, when the bladder is expanded, the perforator vessel is primarily loaded in tension. Moreover, clearance space is provided around a perimeter of the perforator vessel, which may merely be in incidental contact with the bladder (e.g., in occasional contact or not in contact at all), allowing for expansion and growth of the overlying tissue and the perforator vessel, without constricting blood flow through the perforator vessel.

This expander and associated techniques increase the tissue surface area supplied by that vascular pedicle and its flow territory, making the expanded territory available to be used as an island flap, as opposed to the soft tissue areas expanded by conventional expanders that do not support increased blood flow through an isolated perforator vessel.

Advantageously, expanders according to the invention permit increase in both the diameter and length of the perforator vessel, permitting harvesting and use of the enlarged vessel and its expanded vascular territory for use as a free flap, or simply harvesting of the vessel for relocation as a vascular graft.

FIG. 3 is a schematic depiction of before and after sectional views of expanded tissue. The view on left shows an area of tissue supplied by a perforator vessel. The view on the right shows the result of perforator vessel and territory expansion by use of a tissue expander in accordance with the invention. The expansion not only increases the dimensions of the flap and perforator vessel length, but also increases the diameter of the perforator vessel and associated blood flow.

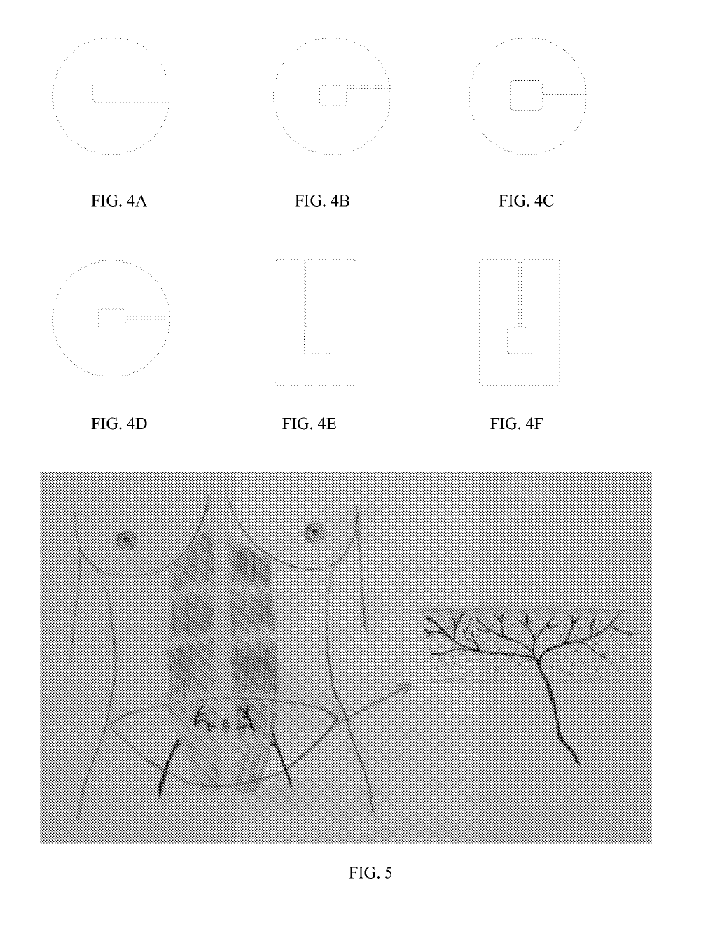

While U-shaped tissue expanders with various arms and reinforcement features have been described with some degree of particularity above, it will be apparent to those skilled in the art that expanders of alternative configurations can be provided to achieve similar benefits. By way of example only, FIGS. 4A-4D depict generally C-shaped bladders. FIG. 4A has a circular profile with a constant width, symmetrical recess. FIG. 4B has a similar circular profile, with a slightly asymmetrical bladder configuration. The arms of the generally C-shaped bladder form a relatively narrow entrance recess connecting to an enlarged end of the recess at the common junction portion. The vessel is nested in the enlarged end of the recess during the expansion period. FIG. 4C is similar to FIG. 4B, but symmetrical about the longitudinal axis of the recess.

FIG. 4D is substantially similar to FIG. 4C, but has a relatively smaller end of the recess. FIGS. 4E and 4F are generally U-shaped, the former with the asymmetric recess of FIG. 4B and the latter with the symmetric recess of FIG. 4C.

In another application, tissue expanders in accordance with the invention can be used advantageously to expand some commonly-used perforator flaps, thereby increasing the number and suitability of donor sites. For example, there are situations where the vascular pedicles typically used to supply island flaps (e.g., Deep Inferior Epigastric Artery Perforator (DIEP) flaps) are inadequate, thereby negating the use of these flaps. See FIG. 5.

Tissue expanders according to the instant invention can be used to increase the size and length of the perforator vessel, thereby making the use of the vessel and its territory a flap option.

Moreover, as discussed briefly hereinabove, these tissue expanders can be used to increase the diameter and length of small perforating vessels sufficiently to allow their use as vascular grafts. Many vascular diseases, such as coronary artery disease, are often treated by replacing or bypassing diseased vessels with vessels harvested from a remote area. These donor vessels can be in short supply or their harvest can often result in morbidity. These tissue expanders can be used to create new sources of donor vessels for bypass procedures. A small perforator vessel can be enlarged sufficiently to allow it to be used as a bypass graft. This allows numerous perforator vessels to become donor vessel candidates and harvested without concern for rejection.

While certain objects and features of various aspects and embodiments of the invention have been depicted and described in the schematic drawings presented herein, it should be understood that the drawings are not necessarily to scale, emphasis instead generally being placed upon illustrating the principles of the invention. Moreover, while certain exemplary and preferred embodiments have been presented, it will be apparent to those skilled in the art that various combinations and permutations of features are contemplated and should be considered to be part of the invention. Numerical values, ranges, materials, and other specific examples are meant to describe certain embodiments only, and should not be considered limiting. The scope of the invention is defined by the claims, as well as the full range of equivalents.

1. A tissue expander adapted for increasing overlying tissue surface area and size of an associated perforator vessel, the tissue expander comprising:
an inflatable bladder adapted to be disposed subcutaneously and forming a recess for receiving therein in incidental contact a perforator vessel; and
a port in fluidic communication with an interior volume of the bladder.

2. The tissue expander of claim 1, wherein the bladder comprises a flexible biocompatible material.

3. The tissue expander of claim 2, wherein the flexible biocompatible material comprises an elastomer.

4. The tissue expander of claim 1, wherein the bladder is adapted to inflate preferentially in a first zone relative to a second zone.

5. The tissue expander of claim 4, wherein the first zone comprises a first wall thickness and the second zone comprises a greater wall thickness.

6. The tissue expander of claim 4, wherein the first zone comprises a first durometer and the second zone comprises a greater durometer.

7. The tissue expander of claim 4, wherein the second zone is disposed along at least a portion of the recess.

8. The tissue expander of claim 4, wherein the second zone is disposed along at least a portion of a perimeter of the bladder remote from the recess.

9. The tissue expander of claim 1, wherein the bladder is generally U-shaped or generally V-shaped or generally C-shaped.

10. The tissue expander of claim 9, wherein the recess is formed by a pair of arms extending from a common junction portion.

11. The tissue expander of claim 10, wherein the arms are substantially parallel.

12. The tissue expander of claim 1, wherein the recess is sized to receive a perforator vessel in a region having a width of at least about 1 mm.

13. The tissue expander of claim 1, wherein the port is disposed on a wall of the bladder.

14. The tissue expander of claim 1, wherein the port is adapted to be disposed remotely from the bladder and is connected thereto with a tube.

15. The tissue expander of claim 1, wherein at least a portion of an exterior of the bladder comprises at least one of a smooth surface and a textured surface and a combination thereof.

16. The tissue expander of claim 1, wherein the bladder comprises a length greater than a width and wherein the recess comprises a slot formed along a portion of a longitudinal centerline of the bladder.

17. The tissue expander of claim 16, wherein the bladder is substantially symmetrical about a plane passing through the longitudinal centerline.

18. The tissue expander of claim 1, wherein the bladder comprises contoured edges.

19. The tissue expander of claim 1, further comprising at least one of:
a support disposed along at least a portion of one side of the bladder; and
a retainer adapted to be secured to adjacent body structure when the bladder is implanted, to reduce a likelihood of migration of the bladder.

20. A method of expanding tissue for use by a patient, the method comprising the steps of:
implanting temporarily a tissue expander beneath a locus of skin and subcutaneous tissue supplied by an associated perforator vessel, wherein the perforator vessel is received at least partially in a recess formed in the tissue expander; and
incrementally increasing an internal volume of the tissue expander over time to increase overlying tissue surface area and size of the associated perforator vessel.

21. The method of claim 20, wherein increase of the size of the associated perforator vessel is incidental to increase of the surface area of the overlying tissue.

22. The method of claim 20, wherein the tissue expander comprises:
an inflatable bladder; and
a port in fluidic communication with an interior volume of the bladder.

23. The method of claim 22, wherein the volume increasing step comprises adding sterile fluid to the internal volume of the bladder via the port.

24. The method of claim 22, wherein the bladder comprises a flexible biocompatible material.

25. The method of claim 24, wherein the flexible biocompatible material comprises an elastomer.

26. The method of claim 22, wherein the volume increasing step inflates the bladder preferentially in a first zone relative to a second zone.

27. The method of claim 26, wherein the first zone comprises a first wall thickness and the second zone comprises a greater wall thickness.

28. The method of claim 26, wherein the first zone comprises a first durometer and the second zone comprises a greater durometer.

29. The method of claim 26, wherein the second zone is disposed along at least a portion of the recess.

30. The method of claim 26, wherein the second zone is disposed along at least a portion of a perimeter of the bladder remote from the recess.

31. The method of claim 22, wherein the bladder is generally U-shaped or generally V-shaped or generally C-shaped.

32. The method of claim 31, wherein the recess is formed by a pair of arms extending from a common junction portion.

33. The method of claim 32, wherein the arms are substantially parallel.

34. The method of claim 20, wherein the recess is sized to receive a perforator vessel in a region having a width of at least about 1 mm.

35. The method of claim 22, wherein the port is disposed on a wall of the bladder.

36. The method of claim 22, wherein the port is disposed remotely from the bladder and is connected thereto with a tube.

37. The method of claim 22, wherein at least a portion of an exterior of the bladder comprises at least one of a smooth surface and a textured surface and a combination thereof.

38. The method of claim 22, wherein the bladder comprises a length greater than a width and wherein the recess comprises a slot formed along a portion of a longitudinal centerline of the bladder.

39. The method of claim 38, wherein the bladder is substantially symmetrical about a plane passing through the longitudinal centerline.

40. The method of claim 22, wherein the bladder comprises contoured edges.

41. The method of claim 22, wherein the expander further comprises at least one of:
a support disposed along at least a portion of one side of the bladder; and
a retainer adapted to be secured to adjacent body structure when the bladder is implanted, to reduce a likelihood of migration of the bladder.

42. The method of claim 20, further comprising the steps of:
removing the tissue expander;
harvesting at least a portion of the expanded tissue and the associated perforator vessel as a free flap;
relocating the free flap to another location of the patient's body; and
attaching the associated perforator vessel to vasculature of the patient.

43. The method of claim 20, further comprising the steps of:
removing the tissue expander;
harvesting at least a portion of the associated perforator vessel;
relocating the harvested vessel to another location of the patient's body; and
attaching the harvested vessel as a vascular graft to vasculature of the patient.

44. The method of claim 20, further comprising the steps of:
removing the tissue expander;
harvesting at least a portion of the expanded tissue and the associated perforator vessel as an island flap; and
relocating the island flap to a proximate location of the patient's body.
